(19) United States
(12) Patent Application Publication
(10) Pub. No.: US 2023/0109065 A1
(43) Pub. Date: Apr. 6, 2023

(54) METHODS FOR DIAGNOSIS AND PREDICTION OF GENETIC DISEASES AND PHENOTYPES FROM LGD MUTATIONS

(71) Applicant: The Trustees of Columbia University in the City of New York, New York, NY (US)

(72) Inventors: Andrew CHIANG, New York, NY (US); Dennis VITKUP, New York, NY (US); Jonathan CHANG, Los Altos, CA (US); Jiayao WANG, New York, NY (US)

(21) Appl. No.: 17/935,957

(22) Filed: Sep. 28, 2022

Related U.S. Application Data
(60) Provisional application No. 63/251,088, filed on Oct. 1, 2021.

(51) Int. Cl. C12Q 1/6883

(52) U.S. Cl. CPC C12Q 1/6883; C12Q 1/6869; C12Q 2600/156; C12Q 2600/158

(57) ABSTRACT

It was discovered that, for individuals with certain types of mutations, clinical outcomes or phenotypes can be very accurately predicted. For example, for an individual with autism harboring a de novo LGD mutation, the patient’s IQ, behavioral phenotypes, and motor/movement phenotypes, and the severity of autism can be predicted. For these LGD mutations, due to a mRNA surveillance mechanism called NMD (nonsense-mediated decay), it was discovered that clinical outcomes and phenotypes are strongly correlated with the expression intensity of the exon harboring the mutation. A method/model was developed, which is called PDS (phenotype dosage sensitivity), to predict phenotypes based on this observation, and the model is able to predict phenotypes at a much higher level of accuracy not previously possible. This disclosure is the first to link LGD mutations and clinical phenotypes in this manner.

### CROSS-REFERENCE TO RELATED APPLICATION

This application claims benefit of U.S. Provisional Pat. Application No. 63/251,088, filed Oct. 1, 2021, the entire contents of which are hereby incorporated by reference as if fully set forth herein.

### GOVERNMENT STATEMENT

This invention was made with government support under grants LM007079 and GM082797 awarded by the National Institutes of Health. The government has certain rights in the invention.

### BACKGROUND

Recent advances in neuropsychiatric genetics [1-4] and, specifically, in the study of autism spectrum disorders (ASD) [5-8] have led to the identification of multiple genes and specific cellular processes that are affected in these diseases [5, 6, 8-10]. However, phenotypes associated with ASD vary considerably across autism probands [11-14], and the nature of this phenotypic heterogeneity is not well understood [15, 16]. Despite the complex genetic architecture of ASD [17-22], a subset of cases from simplex families, i.e. families with only a single affected child among siblings, are known to be strongly affected by de novo mutations with severe deleterious effects [8, 23, 24]. Interestingly, despite their less complex genetic architecture, simplex autism cases often display as much phenotypic heterogeneity as more general ASD cohorts [25-27]. This provides an opportunity for an in-depth exploration of the etiology of the autism phenotypic heterogeneity using accumulated phenotypic and genetic data.

### BRIEF DESCRIPTION OF THE DRAWINGS

The present embodiments are illustrated by way of example, and not by way of limitation, in the figures of the accompanying drawings.

The following figures are illustrative only, and are not intended to be limiting

FIG. 1 are graphs showing the relationship between the relative expression of exons harboring LGD mutations and the corresponding decreases in probands’ intellectual phenotypes.

FIG. 2 is an illustration of the method used to estimate phenotypic sensitivity to changes in the dosage of a gene.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed through the present specification unless otherwise indicated.

As used herein and in the appended claims, the singular forms “a,” “an,” and “the” include plural references unless the content clearly dictates otherwise.

The term “about” as used herein means approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

The terms “autistic spectrum disorder” or “ASD” refers to autism and similar disorders. Examples of ASD include disorders listed in the Diagnostic and Statistical Manual of Mental Disorders (DSM-IV). Examples include, without limitation, autistic disorder, Asperger’s disorder, pervasive developmental disorder, childhood disintegrative disorder, and Rett’s disorder. Known ASD diagnostic screenings methods include, without limitation: Modified Checklist for Autism in Toddlers (M-CHAT), the Early Screening of Autistic Traits Questionnaire, and the First Year Inventory; the M-CHAT and its predecessor CHAT on children aged 18-30 months, Autism Diagnostic Interview (ADI), Autism Diagnostic Interview-Revised (ADI-R), the Autism Diagnostic Observation Schedule (ADOS) The Childhood Autism Rating Scale (CARS), and combinations thereof. Known symptoms, impairments, or behaviors associated with ASD include without limitation: impairment in social interaction, impairment in social development, impairment with communication, behavior problems, repetitive behavior, stereotypy, compulsive behavior, sameness, ritualistic behavior, restricted behavior, self-injury, unusual response to sensory stimuli, impairment in emotion, problems with emotional attachment, impaired communication, and combinations thereof.

The terms “diagnosing” or “diagnose” refer to detecting and identifying a disease/disorder in a subject. The term may also encompass assessing or evaluating the disease/disorder status (severity, classification, progression, regression, stabilization, response to treatment, etc.) in a patient. The diagnosis may include a prognosis of the disease/disorder in the subject.

The term “mutation” refers to one or more changes to the sequence of a DNA nucleotide sequence or a protein amino acid sequence relative to a reference sequence, usually a wild-type sequence. A mutation in a DNA sequence may or may not result in a corresponding change to the amino acid sequence of an encoded protein. A mutation may be likely gene disrupting (LGD) or loss of function (LoF) i.e. any mutation that leads to nonsense-mediated decay. LGD mutations include nonsense, frameshift, and splice-site mutations. A mutation may be a point mutation, i.e. an exchange of a single nucleotide and/or amino acid for another. Point mutations that occur within the protein-coding region of a gene’s DNA sequence may be classified as a silent mutation (coding for the same amino acid), a missense mutation (coding for a different amino acid), and a nonsense mutation (coding for a stop which can truncate the protein). A mutation may also be an insertion, i.e. an addition of one or more extra nucleotides and/or amino acids into the sequence. Insertions in the coding region of a gene may alter splicing of the mRNA (splice site mutation), or cause a shift in the reading frame (frameshift), both of which can significantly alter the gene product. A mutation may also be a deletion, i.e. removal of one or more nucleotides and/or amino acids from the sequence. Deletions in the coding region of a gene may alter the splicing and/or reading frame of the gene. A mutation may be spontaneous, induced, naturally occurring, or genetically engineered.

The term “phenotype dosage sensitivity” refers to the slope of the fitted regression between the relative expression of a target exon and the effect of a mutation. PDS is a parameter which quantifies the relationship between changes in a gene’s dosage and changes in a given disease phenotype.

A “phenotype” is any observable, detectable or measurable characteristic of an organism, such as a condition, disease, disorder, trait, behavior, biochemical property, metabolic property or physiological property.

The terms “predicting” or “prediction” refers to the forecasting of likely or expected phenotypes, traits, symptoms, conditions, or survival associated with an illness or condition. Phenotypes or traits can be predicted that may not be directly related to the disorder/indication at hand. For example, the disclosed method can predict IQ and more general behavioral test scores, in addition to severity of ASD symptoms like repetitive behavior.

The term “relative expression” refers to the exon expression relative to the expression of other exons of the same gene. Due to alternative splicing and isoforms, an exon may not be expressed in a gene. Exon expression is calculated from the total amount of mRNA containing the exon expressed.

“Sample” or “biological sample” means biological material isolated from a subject. The biological sample may contain any biological material suitable for sequencing the desired genes or exons, and may comprise cellular material from the subject. The sample can be isolated from any suitable biological fluid such as, for example, blood, blood plasma, blood serum, cheek swabs, or tissue, or tissue homogenate.

“Sequencing” as used herein refers to any method to obtain sequence data obtained from nucleic acids from an individual. Such methods include, but are not limited to, whole genome sequencing, exome sequencing, transcriptome sequencing, cDNA library sequencing, kinome sequencing, metabolomic sequencing, microbiome sequencing, and the like.

The term “subject” as used herein refers to an individual. For example, the subject is a human. The term does not denote a particular age or sex. Thus, adult and newborn subjects, whether male or female, are intended to be covered. As used herein, patient or subject may be used interchangeably and can refer to a subject afflicted with a disease or disorder.

The terms “treat”, “treating” or “treatment of” as used herein refers to providing any type of medical management to a subject. Treating includes, but is not limited to, administering a composition to a subject using any known method for purposes such as curing, reversing, alleviating, reducing the severity of, inhibiting the progression of, or reducing the likelihood of a disease, disorder, or condition or one or more symptoms or manifestations of a disease, disorder or condition. For example, treatment for an ASD may range from behavioral interventions to dietary approaches to medications for enhancing function.

### DETAILED DESCRIPTION

### Overview

An analysis was performed, focusing on severely damaging, so-called likely gene-disrupting (LGD) mutations, which include nonsense, splice site, and frameshift variants. Genetic and phenotypic data collected in the Simons Simplex Collection (SSC) [28] was explored, and then the results are validated using an independent ASD cohort from the Simons Variation in Individuals Project (VIP) [29].

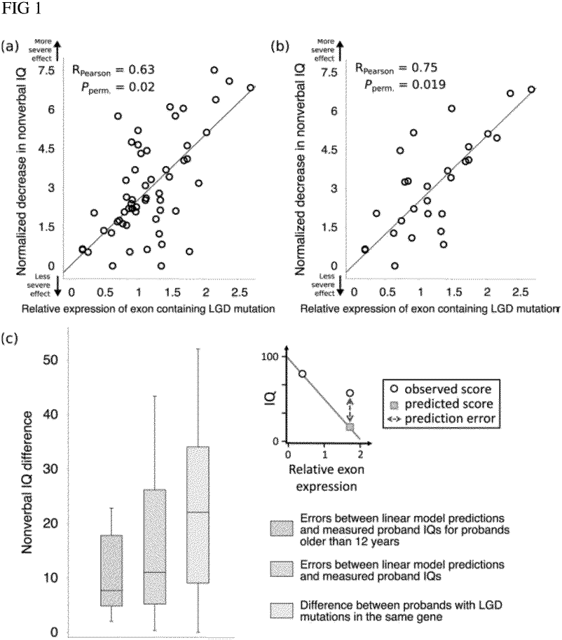

The effects of LGD mutations are investigated on cognitive and other important ASDrelated phenotypes, including adaptive behavior, motor skills, communication, and coordination. These analyses allowed the understanding of how the exon-intron structure of human genes contributes to observed phenotypic heterogeneity. The quantitative relationships between changes in gene dosage induced by nonsense-mediated decay (NMD) and the phenotypic effects of LGD mutations was explored. To that end, a new genetic parameter was introduced, which quantifies how changes in a gene’s dosage affect specific autism phenotypes (FIG. 1). Finally, it was described how simple regression models of gene dosage can explain a substantial fraction of the phenotypic heterogeneity in the analyzed simplex ASD cohorts.

### Sequencing and Arrays

In some embodiments, sequencing libraries comprising sequenceable material are made from the genetic material from each sample prior to sequencing, using any suitable technique known to one of ordinary skill in the art, including the fragmentation, tagging of genetic material with sequencing adaptors to provide sequenceable material, and may optionally include any subsequent amplification of the genetic material (e.g., DNA) comprising the genetic sample. In some embodiments, hybridization and hybrid capture are used to create the sequence library.

Any suitable technique for sequencing exome DNA from the samples can be used in various embodiments of the present methods. Apparatuses and materials for carrying out such sequencing techniques are well-known in the art, and are commercially available. For example, suitable sequencing machines and protocols are available from Illumina, Inc. of San Diego, Calif. as the Illumina MiSeq or Illumina HiSeq 2500. The sequencing results can be in any standard output format that is suitable for storage and retrieval in a database, and/or for further analysis, as are well-known to one of ordinary skill in the art; for example, in Picard BAM format. In some embodiments, the output is de-multiplexed, for example so that a single Picard BAM file corresponds to a single identified (e.g., barcoded) sample. In one embodiment, genetic material derived from multiple genetic samples is sequenced in a high throughput manner, in order to take advantage of economies of scale. In certain embodiments, sequencing reactions are conducted at a low-volume, e.g., at a volume less than that used for standard sequencing reactions. For example, a low-volume sequencing reaction can be about ½, ⅓, ¼, ⅕, ⅙, ⅐, ⅛, ⅑, ⅒, 1/12, 1/15, 1/20, 1/25 or 1/30 the standard volume for a given reaction.

In some embodiments, the sequencing performed is RNA-seq to determine the gene expression in a subject. RNA-seq is commonly used for identification, classification, and quantification of gene expression within subjects. Apparatuses and materials for carrying out such sequencing techniques are well-known in the art, and are commercially available. In some embodiments, 200 ng of total RNA is used from each sample as the starting material. This method uses oligo dT beads to select poly-A mRNA from the total RNA sample. The selected RNA is then heat fragmented and randomly primed before cDNA synthesis from the RNA template. The resultant cDNA then goes through Illumina library preparation (end repair, base ‘A’ addition, adapter ligation, and enrichment) using Broad designed indexed adapters for multiplexing of samples. In some embodiments, sequencing is performed on Illumina HiSeq 2000 instruments, with sequence coverage to a minimum of 50 M reads (corresponding to a minimum of 25 M 76 bp paired-end reads). The sequencing results can be in any standard output format that is suitable for storage and retrieval in a database; for example, in SRA submitted files with a binary alignment map for each sequence.

In some embodiments, reads need to be filtered to produce gene and exon level read count and gene level RPKM values. Filtering includes: (1) reads are uniquely mapped; (2) reads must have proper pairs; (3) alignment distance must be <=6; (4) reads must be contained 100% within exon boundaries. Reads overlapping introns were not counted. For exon read counts, if a read overlapped multiple exons, then a fractional value equal to the portion of the read contained within that exon was allotted. Several additional quality control metrics can be applied to RNA-seq samples to determine inclusion. All samples with fewer than 10 million mapped reads are removed, and sample outliers are identified using a correlation-based statistic. For all processing replicates (the same sample sequenced twice), only the sample with the greater number of reads was retained for inclusion in the final analysis set.

In some embodiments, microarrays are used for detecting one or more LGD mutations. A microarray is a multiplex lab-on-a-chip. It is a 2D array on a solid substrate (usually a glass slide or silicon thin-film cell) that assays large amounts of biological material using high-throughput screening miniaturized, multiplexed and parallel processing and detection methods. Microarrays are known in the art and available commercially from companies such as Affymetrix, Agilent, Applied Microarrays, Arrayit, Illumina, and others. The array contains probes complementary to at least one single mutation, preferably probes are included for hybridization to the LGD mutations.

It will be readily apparent to one skilled in the art that the exact formulation of probes on an array is not critical as long as the user is able to select probes for inclusion on the array that fulfill the function of hybridizing to the mutations. The array can be modified to suit the needs of the user. Thus, analysis of the array can provide the user with information regarding the number and/or presence of LGD mutations in a given sample.

### Expression Parameters

### Gene Expression Changes Due to Lgd Variants in GTEx

To quantify altered gene expression due to an LGD variant, the changes in expression (Δx) compared to wild type as a combined effect of allele-specific expression (AE), alternative splicing (AS), and nonsense-mediated decay (NMD) are considered. To account for AE, it is reasoned that only a fraction of total mRNA would be transcribed from each allele. To account for alternative splicing, it is reasoned that transcripts would be spliced into multiple transcript isoforms, only some of which would retain the exon with the truncating mutation. Finally, it is assumed that nonsense-mediated decay is an imperfect degradation process, in which some fraction of LGD-containing mRNA escapes NMD. Formally, we represented a change in expression as:

$$Δ x = f ⋅ ∈ ⋅ {x}_{e x o n}$$

where the parameter f (ranging from 0 to 1) quantifies the fraction of total transcription from the allele harboring the LGD variant, the parameter ∈ (ranging from 0 to 1) quantifies NMD efficiency, and Xₑₓₒₙ represents the wildtype expression level of transcripts with the LGD-containing exon, i.e. transcripts susceptible to NMD.

Because only post-NMD expression levels are experimentally observed, the relationship between measured and wild-type expression levels can be expressed as:

$${{x}^{′}}_{e x o n} = {x}_{e x o n} - Δ x$$

where x’ₑₓₒₙ represents the experimentally observed expression. Combining the above equations, the effects of NMD in terms of x’ₑₓₒₙ are express:

$$Δ x = \frac{f ⋅ ∈}{1 - f ⋅ ∈} ⋅ {{x}^{′}}_{e x o n}$$

In order to estimate Δx for each gene, it is needed to infer the parameters f and ∈, which quantify AE and NMD efficiency respectively. As described in the following sections, these parameters are inferred probabilistically by fitting appropriate distributions. Separate analyses are performed for each tissue.

### Correlation Between Changes in Gene Dosage and Phenotypic Effects

Human genes likely differ in their contributions to cognitive phenotypes. Therefore, for each gene with multiple LGD mutations in SSC, the IQ phenotype’s sensitivity is estimated to changes in gene dosage (i.e. phenotype dosage sensitivity or PDS). In a specific embodiment, least-squares linear regressions are used, regressing the observed phenotypic effects (y), defined as the difference between the average neurotypical IQ (100) and the proband’s IQ, against the relative expression of LGD targeted exons

$$\left({x}_{r e l} = \frac{{x}_{e x o n}}{{x}_{g e n e}}\right) .$$

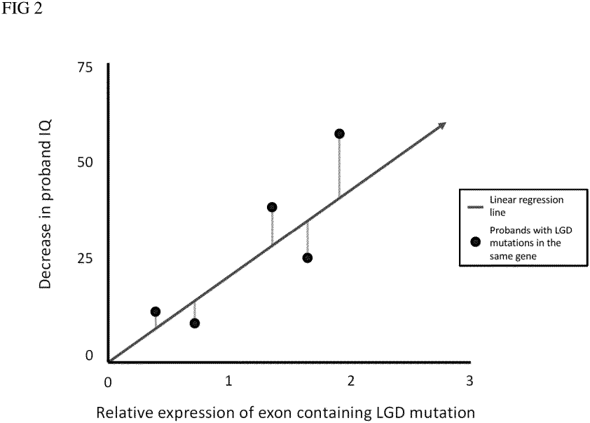

In each regression, it is assumed that normal (wild type) gene dosage corresponds to a neurotypical IQ (100), and therefore in some embodiments the y-intercept is fixed at 0. The slope (s) of the fitted least-squares regression line provided an estimate of the phenotypic sensitivity to gene dosage (FIG. 2).

As shown in FIG. 2, each gene with multiple truncating mutations in SSC, least-squares linear regression is used to estimate the phenotypic sensitivity. Phenotypic sensitivity is defined as the slope of the fitted regression between the relative expression of the target exon

$$( \frac{{x}_{e x o n}}{{x}_{g e n e}} )$$

and the effect of the mutation, i.e. the corresponding proband’s IQ compared to the average neurotypical value (100). Each blue point in the figure represents a proband with an LGD mutation in the same gene. The x-axis position indicates the relative expression the targeted exon. The y-axis position indicates the observed effect of the mutation on IQ. The red line shows the least-squares regression line.

### Treatments and Therapies

Certain embodiments describe treatment for phenotypes diagnosed by phenotype dosage sensitivity. Currently, no treatment has been shown to cure ASD, but several interventions have been developed and studied for use with young children. These interventions may reduce symptoms, improve cognitive ability and daily living skills, and maximize the ability of the child to function and participate in the community. The differences in how ASD affects each person means that people with ASD have unique strengths and challenges in social communication, behavior, and cognitive ability. In certain embodiments, treatments are usually multidisciplinary, may involve parent-mediated interventions, and target the child’s individual needs. Also, the gene expression dosage decrease estimations described herein can be used to personalize treatment by estimating the correct amount of a therapeutic to dose based on level of gene expression dosage.

Behavioral intervention strategies have focused on social communication skill development—particularly at young ages when the child would naturally be gaining these skills—and reduction of restricted interests and repetitive and challenging behaviors. In certain embodiments, occupational and speech therapy may be helpful, as could social skills training and medication in older children. The best treatment or intervention can vary depending on an individual’s age, strengths, challenges, and differences. Behavior approaches can be, but not limited to; applied behavior analysis, discrete trail training, early intensive behavioral intervention, early start denvor model, pivotal response training, and verbal behavior intervention. Assistive technology, such as communication boards, electronic tablets, and Picture Exchange Communication Systems, can be used as therapies for patients. Further behavior approaches can be occupational therapy, social skills training, and speech therapy.

Currently, there is no medication that can cure autism spectrum disorder (ASD) or all of its symptoms. But some medications can help treat certain symptoms associated with ASD, especially certain behaviors. In certain embodiments, medications prescribed can be selective serotonin re-uptake inhibitors, tricyclics, psychoactive or anti-psychotic medications, stimulants, anti-anxiety medications, or anticonvulsants. Healthcare providers usually prescribe a medication on a trial basis to see if it helps. Some medications may make symptoms worse at first or take several weeks to work. Healthcare providers may have to try different dosages or different combinations of medications to find the most effective plan.

Further background and supporting information for the embodiments described and claimed herein is provided in Chiang et al., Mol. Psychiatry, (2021) 26:1685-1695, which is incorporated herein in its entirety.

### REFERENCES

1. Rivas, M. A. et al. Human genomics. Effect of predicted protein-truncating genetic variants on the human transcriptome. Science 348, 666-669 (2015).
2. Quinlan, A. R. & Hall, I. M. BEDTools: a flexible suite of utilities for comparing genomic features. Bioinformatics (Oxford, England) 26, 841-842, doi:10.1093/bioinformatics/btq033 (2010).
3. Li, H. & Durbin, R. Fast and accurate short read alignment with Burrows-Wheeler transform. Bioinformatics (Oxford, England) 25, 1754-1760, doi:10.1093/bioinformatics/btp324 (2009).
4. Pickrell, J. K. et al. Understanding mechanisms underlying human gene expression variation with RNA sequencing. 464, 768, doi:10.1038/nature08872 (2010).
5. Skelly, D. A., Johansson, M., Madeoy, J., Wakefield, J. & Akey, J. M. A powerful and flexible statistical framework for testing hypotheses of allele-specific gene expression from RNA-seq data. Genome Research 21, 1728-1737 (2011).
6. Gelman, A. Prior distributions for variance parameters in hierarchical models (comment on article by Browne and Draper). Bayesian Anal. 1, 515-534, doi:10.1214/06-BA117A (2006).

What is claimed is:

1. A method comprising: i) collecting a sample from a subject, ii) sequencing nucleic acids from the sample, iii) identifying mutations in one or more exons of the nucleic acids, iv) calculating a relative expression for each exon containing mutations, v) diagnosing or predicting one or more potential phenotypes by fitting the relative expression into a phenotype dosage sensitivity (PDS) regression model, and vi) optionally, if a PDS is unknown for an exon, then calculating a PDS linear regression model for said exon.

2. The method of claim 1, wherein the sample comprises blood, blood plasma, blood serum, urine, tissue, or tissue homogenate.

3. The method of claim 1, wherein the sequencing comprises one or more of the following: whole genome sequencing, whole-exome sequencing, targeted sequencing, RNA-seq, microarrays, restriction fragment length polymorphism identification (RFLPI), random amplified polymorphic detection (RAPD), amplified fragment length polymorphism detection (AFLPD), or polymerase chain reaction (PCR).

4. The method of claim 1, wherein the mutations comprise one or more of the following: nonsense variants, frameshift, indels, splice acceptor variants, splice donor variants, loss of function (LoF) or any other likely gene-disrupting (LGD) mutations.

5. The method of claim 4, wherein non-LGD and non-LoF mutations in an exon are removed from calculating relative expression of said exon.

6. The method of claim 1 wherein the relative expression is calculated from a mutation’s location in a gene.

7. The method of claim 1, wherein the PDS regression model is calculated from an exon-level expression dataset and a paired mutations and phenotypes dataset.

8. The method of claim 7, wherein the PDS regression model is calculated using normalized phenotype effects of each mutation.

9. The method of claim 8, wherein phenotype effects are normalized based on a subject’s sex.

10. The method of claim 1, wherein the potential phenotype comprises one or more of the following: IQ, behavioral phenotypes, motor phenotypes, or severity of a disorder.

11. A method for diagnosing a genetic disorder in a subject in need comprising: i) collecting a sample from a subject, ii) sequencing nucleic acids, iii) identifying mutations in one or more exons, iv) calculating a relative expression for each exon containing mutations, v) diagnosing one or more potential phenotypes by fitting the relative expression into a phenotype dosage sensitivity (PDS) regression model, and vi) optionally, if a PDS is unknown for an exon, then calculating a PDS linear regression model for said exon.

12. The method of claim 11, wherein the genetic disorder comprises an autistic spectrum disorder (ASD) or autism.

13. The method of claim 11, further comprising administering a treatment of one or more genetic disorders.

14. The method of claim 13, wherein the treatment comprises a personalized therapy for the genetic disorder.
